(19) United States
(12) Patent Application Publication
(10) Pub. No.: US 2016/0368842 A1
(43) Pub. Date: Dec. 22, 2016

(54) CONTINUOUS PROCESS FOR PRODUCING AN ETHYLENE GLYCOL STREAM

(71) Applicant: BIOCHEMTEX S.P.A., Tortona (AL) (IT)

(72) Inventors: Marco BERNARDI, Bologna (IT); Irene BOZZANO, Genova (IT); Daniela TODARO, Milano (IT)

(73) Assignee: Biochemtex S.p.A., Tortona (IT)

(21) Appl. No.: 15/120,756

(22) PCT Filed: Feb. 13, 2015
(86) PCT No.: PCT/EP2015/053053

§ 371 Date: Aug. 23, 2016

(30) Foreign Application Priority Data
Feb. 25, 2014 (IT) MI2014A 000287

(51) Int. Cl. C07C 29/132; C07C 29/60

(52) U.S. Cl. CPC C07C 29/132; C07C 29/60

(57) ABSTRACT

It is disclosed a continuous process for producing a polyols stream comprising ethylene glycol and propylene glycol, wherein a liquid sugar stream comprising water and at least a monomeric sugar is introduced into a first reaction zone and subjected to a first reaction, which is a hydrogenation reaction of the at least a monomeric sugar, at a first catalytic conditions and in the presence of a first hydrogen gas to produce an hydrogenated mixture comprising at least a sugar alcohol. At least a portion of the hydrogenated mixture and at least a portion of the first hydrogen gas are then transferred to a second reaction zone and subjected to a second reaction, which is a hydrogenolysis reaction of the at least a sugar alcohol, at second catalytic conditions to produce a hydrogenolysis mixture comprising at least a polyol. The second reaction occurs in the presence of a second hydrogen gas, wherein the second hydrogen gas comprises the portion of the first hydrogen gas which has been transferred to the second reaction. The first reaction is conducted at a first reaction pressure which is greater than or equal to the second reaction pressure, and the hydrogen gas is then released at the end of the conversion process of the second reaction zone at a discharge pressure which is less than the pressure of the first reaction. In this way, the transferring of at least a portion of the hydrogenated mixture and at least a portion of the first hydrogen gas from the first reaction zone to the second reaction zone may be done without the use of any pumping system, specifically of a pumping system which is capable of pumping a pressurized liquid/gas mixture.

### FIELD OF THE INVENTION

The present invention relates to a continuous process for converting monomeric sugars to polyols, such as ethylene glycol and propylene glycol, which can be scaled up to industrial scale.

### BACKGROUND

Conversion of biomass has attracted significant attention as a key technology for replacing oil as the source of renewable fuels and chemicals. Lignocellulose is the most abundant biomass resource, and is not digestible for human beings, which is an advantage over sugars and starch since the use of edible carbohydrates has competed with the food production. Therefore, lignocellulose is one of the most attractive biomass resources in nature, available at a very low cost.

For effectively replacing fossil oil, renewable fuels and chemicals have not only to meet the technical specification in terms of performance, but they must be produced at a competitive cost with the oil derived competitors.

Ethylene glycol and propylene glycol are two oil-derived polyols which are widely used as starting materials in the polymer chemistry. Many processes have been developed for converting water soluble and insoluble sugar sources to polyols. Nevertheless, even if the conversion chemistry is well known, none of prior art processes has been found real industrial applicability so far. Some of the prior art methods demonstrate the conversion of synthetic sugars to light polyols; in the case of conversion of lignocellulose derived sugars, the prior art processes produce polyols mixtures having poor properties for finding real use and in general are too much expensive for competing with oil derived polyols. One of the main issue is related to the fact that it is difficult to control the competitive reaction pathways, therefore the stream derived from the ligno-cellulosic feedstock is a mixture which usually comprises many compounds.

It is known in the art that ethylene glycol and propylene glycol may be obtained from the monomeric sugars, such as glucose and xylose, by means of a conversion process comprising an hydrogenation step of the monomeric sugars to produces sugar alcohols, such as xylitol and sorbitol, followed by a hydrogenolysis step of the sugar alcohols to produce polyols comprising ethylene glycol and propylene glycol.

As exemplary prior art of conversion of biomass-derived xylose to xylitol, in H M Baudel et al., “Xylitol production via catalytic hydrogenation of sugarcane bagasse dissolving pulp liquid effluents over Ru/C catalyst”, J. Chem. Technol. Biotechnol. 80:230-233 (2005), xylose-rich liquid effluents generated by the acid hydrolysis of sugarcane bagasse for production of dissolving pulp were converted to xylitol, via catalytic hydrogenation over Ru/C. The paper shows that Ruthenium (Ru 2%/C) catalysts are suitable to convert bagasse hydrolysate sugars into polyols, with high selectivity towards xylitol (above 98%), even under mild temperature and hydrogen pressure levels (80° C., 20 atm).

As exemplary prior art of conversion of xylitol to polyols, in J. Sun et al., “Selective hydrogenolysis of biomass-derived xylitol to ethylene glycol and propylene glycol on supported Ru catalysts”, Green Chem., 2011, 13, p.135, the selective hydrogenolysis of biomass-derived xylitol to ethylene glycol and propylene glycol was carried out on different catalysts in the presence of Ca(OH)2. The catalysts included Ru supported on activated carbon (C) and, for comparison, on metal oxides, Al2O3, TiO2, ZrO2 and Mg2AlOx as well as C-supported other noble metals, Rh, Pd and Pt. The hydrogenolysis reaction is conducted by pressurizing the reactor by hydrogen at 40 bar and then heating the reactor at 473° C. The authors show how to control the selectivity of the hydrogenolysis of xylitol to different polyols, comprising ethylene glycol, propylene glycol, glycerol, threitol, arabitol by varying the hydrogen pressure at 473° C. It is highlighted that reaction activity decreases rapidly by reducing the hydrogen pressure.

As known in the art, hydrogenation of monomeric sugars to sugar alcohol is a reaction which can be performed in milder conditions, in terms of hydrogen pressure and temperature, with respect to reaction conditions hydrogenolysis of sugar alcohols to polyols. In particular, according to the teaching of the prior art, the two reactions are carried out separately, the first reaction, i.e. hydrogenation, being conducted at a first reaction pressure lower than the pressure of the second reaction (hygrogenolysis). This is usually considered convenient from an applicative point of view, as it corresponds to a cost reduction of the hydrogenation reactor.

A continuous process for converting monomeric sugars to polyols is required to scale up the process for converting monomeric sugars to polyols to industrial scale. At industrial scale, transferring of pressurized streams, comprising a gas portion and a liquid portion, from a first plant section at a lower pressure as the first reaction pressure to a second plant section at a higher pressure as the second reaction pressure will require to use of large and special pumping systems, which are however expensive and subjected to failure. As an alternative solution, the pressurized stream may be separated into the liquid portion and the gas portion, and then each portion is pressurized and pumped into the second plant section.

This procedure is usually accomplished by preliminary cooling down the pressurized stream prior to separate the liquid and gas portions. However, this separation procedure is not really applicable in a continuous process at industrial scale, and in any case requires supplementary equipments which involve additional operating and investment costs.

The technical problem underlying the present invention is thus that of developing a continuous process to convert monomeric sugars to polyols, such as ethylene glycol and propylene glycol, which can be scaled up to industrial scale so as to overcome the drawbacks previously mentioned with reference to the prior art.

### SUMMARY OF THE INVENTION

This technical problem is solved, according to the invention, by a continuous process for producing a polyols stream comprising ethylene glycol and propylene glycol, wherein the process comprises the steps of:
a. Introducing a first hydrogen gas and a liquid sugar stream, said liquid sugar stream comprising water and at least a monomeric sugar, into a first reaction zone, wherein the first reaction zone contains a hydrogenation catalyst and has a first reaction pressure and a first reaction temperature,
b. Maintaining the liquid sugar stream in the first reaction zone in contact with at least the hydrogenation catalyst for a first reaction time sufficient to produce an hydrogenated mixture comprising at least a sugar alcohol;
c. Transferring at least a portion of the hydrogenated mixture and at least a portion of the first hydrogen gas from the first reaction zone to a second reaction zone, wherein the second reaction zone has a second reaction pressure which is not greater than the first reaction pressure and a second reaction temperature and further contains a hydrogenolysis catalyst capable of converting the sugar alcohol to at least a polyol at the second reaction pressure and the second reaction temperature,
d. Maintaining the at least a portion of the hydrogenated mixture in the second reaction zone in contact with the hydrogenolysis catalyst in the presence of OH− ions and a second hydrogen gas comprising the at least a portion of the first hydrogen gas for a second reaction time sufficient to produce an hydrogenolysis mixture comprising the polyols,
e. Releasing at least a portion of the second hydrogen gas at a discharge pressure, wherein the discharge pressure is not greater than the second pressure, and
f. Recovering at least a portion of the polyols from at least a portion of the hydrogenolysis mixture to form the polyols stream,
wherein the first reaction pressure is greater than the discharge pressure.

It is also disclosed that the step of transferring at least a portion of the hydrogenated mixture and at least a portion of the first hydrogen gas from the first reaction zone to a second reaction zone may occur without the use of a pumping system.

It is further disclosed that the transferring of at least a portion of the hydrogenated mixture and at least a portion of the first hydrogen gas from the first reaction zone to a second reaction zone may occur under the action of a driving force which is the force associated to the difference of the first reaction pressure and the discharge pressure.

It is also disclosed that the first reaction pressure and the second reaction pressure may be both a value in a range selected from the group consisting of 40 bar to 170, 40 bar to 150 bar, 50 bar to 100 bar, and 60 to 80 bar, provided that the second reaction pressure is not greater than the first reaction pressure.

It is further disclosed that the discharge pressure may be a value in a range of 0.1 bar to 30 bar, 0.3 bar to 20 bar, 0.5 bar to 10 bar, 0.5 bar to 4 bar, and 0.5 bar to 2 bar.

It is also disclosed that the process may further comprise the step of separating the at least a portion of the hydrogenolysis mixture and the at least a portion of the second hydrogen gas before releasing the at least a portion of the second hydrogen gas.

It is further disclosed that the separation of the at least a portion of the hydrogenolysis mixture and the at least a portion of the second hydrogen gas may occur in a separation zone, wherein the separation zone has a separation pressure which is less than or equal to the second reaction pressure and greater than the discharge pressure.

It is also disclosed that the at least a portion of the hydrogenolysis mixture and the at least a portion of the second hydrogen gas may be transferred from the second reaction zone to the separation zone without the use of a pumping system

It is further disclosed that the transferring of the at least a portion of the hydrogenolysis mixture and at least a portion of the second hydrogen gas from the second reaction zone to a separation zone may occur under the action of a driving force which is the force associated to the difference of the first reaction pressure and the discharge pressure.

It is also disclosed that the liquid sugar stream and the first hydrogen gas may be mixed together before being inserted in the first reaction zone.

It is further disclosed that the ratio of the total molar amount of monomeric sugars to the molar amount of the first hydrogen gas introduced in the first reaction zone per unit of time may be a value in a range selected from the group consisting of 1:2 to 1:10, 1:3 to 1:8, and 1:4: to 1:6.

It is also disclosed that the second hydrogen gas may further comprise hydrogen added to the second reaction zone from an external hydrogen source.

It is further disclosed that the ratio of the total molar amount of sugar alcohols to the molar amount of the second hydrogen gas introduced in the second reaction zone may be a value in a range selected from the group consisting of 1:2 to 1:10, 1:3 to 1:8, and 1:4: to 1:6.

It is also disclosed that the first reaction time may be a value in a range selected from the group consisting of 20 minutes to 3 h, 25 minutes to 2 h, and 30 minutes to 1 hour.

It is further disclosed that the second reaction time may be a value in a range selected from the group consisting of 15 minutes to 10 h, 20 minutes to 5 h, 25 minutes to 2 h, and 30 minutes to 1 h.

It is also disclosed that the first reaction zone may be located in a first reactor and the second reaction zone may be located in a second reactor.

It is further disclosed that the first reactor and/or the second reactor may be selected from the group consisting of fixed bed reactor, fluidized bed reactor and mechanically mixed reaction system.

It is also disclosed that the first reactor and/or the second reactor may be a fixed bed reactor operated as a trickle bed reactor.

It is further disclosed that the first reaction zone and the second reaction zone may be located in a unique reactor.

It is also disclosed that the unique reactor may be selected from the group consisting of fixed bed reactor, fluidized bed reactor and mechanically mixed reaction system.

It is further disclosed that the unique reactor may be a fixed bed reactor operated as a trickle bed reactor.

It is further disclosed that the separation zone may be located in a separation vessel.

It is also disclosed that the at least a monomeric sugar may comprise at least a compound selected from the group consisting of xylose, glucose and arabinose, or mixture thereof.

It is further disclosed that the at least a monomeric sugar comprises xylose and the percent amount of xylose by weight on a dry matter basis in the liquid sugar stream may be greater than a value selected from the group consisting of 50%, 70%, 80%, 90% and 95%.

It is also disclosed that at least a portion of the monomeric sugars may have been obtained from a liquid stream comprising of solubilized C5 and C6 sugars which has been removed from a ligno-cellulosic biomass feedstock stream

It is further disclosed that the amount of solubilized C5 sugars in the liquid stream may be greater than the amount of solubilized C6 sugars.

It is also disclosed that the at least a sugar alcohol may comprise a compound selected from the group consisting of xylitol, sorbitol and arabitol, or mixture thereof.

It is further disclosed that at least a portion of the ethylene glycol and at least a portion of the propylene glycol in the polyols stream may be separated from polyols stream to produce an ethylene glycol stream comprising ethylene glycol and a propylene glycol stream comprising propylene glycol.

It is also disclosed that the ethylene glycol stream may further comprise at least one diol selected from the group consisting of 1,2-Propylene glycol, 1,2-Butanediol and 1,2-Pentanediol.

It is further disclosed that the ethylene glycol stream may be used for producing a polyester resin.

It is also disclosed that the polyester may comprise acid moieties and that at least 85 mole % of the acid moieties may be derived from terephthalic acid or its dimethyl ester.

It is further disclosed that the polyester resin may be used for producing a polyester bottle.

### DETAILED DESCRIPTION

It is disclosed a continuous process for catalytically converting a liquid sugar stream to a polyols stream comprising ethylene glycol and propylene glycol.

A process for converting a feedstock to a product or products may be run in a continuous or a batch operation. In batch operation, the process occurs in time-sequential steps in batches. A batch of feedstock is introduced into a reaction zone, then the conversion of the feedstock to the product or products takes place, then the product or products are removed from the reaction zone.

In a continuous process, the feedstock is being introduced as a stream into the reaction zone and a stream comprising the product or products is being removed, while the conversion is occurring. The streams may be introduced or removed continuously or discontinuously, with the process considered continuous in both cases.

The liquid sugar stream comprises water and at least a monomeric sugar and is introduced into a first reaction zone and subjected to a first reaction, which is a hydrogenation reaction of the at least a monomeric sugar, at a first catalytic conditions to produce an hydrogenated mixture comprising at least a sugar alcohol. Preferably, the sugar alcohol should be derived from the monomeric sugar. A first hydrogen gas is also introduced in the first reaction zone, as the first reaction occurs in the presence of hydrogen. At least a portion of the hydrogenated mixture and at least a portion of the first hydrogen gas are then transferred to a second reaction zone and subjected to a second reaction, which is a hydrogenolysis reaction of the at least a sugar alcohol, at second catalytic conditions to produce a hydrogenolysis mixture comprising at least a polyol. The second reaction occurs in the presence of a second hydrogen gas, wherein the second hydrogen gas comprises the portion of the first hydrogen gas which has been transferred to the second reaction.

At least a portion of the second hydrogen gas is then released at a discharge pressure which is not greater, preferably lower, that the second reaction pressure and the polyols stream is recovered from at least a portion of the hydrogenolysis mixture.

As already mentioned previously, according to the teaching of the prior art, which does not consider a continuous process for converting a monomeric sugar to a polyol but the two reactions separately, the first reaction is conducted at a first reaction pressure lower than the second reaction pressure. This is usually considered convenient from an applicative point of view, as it corresponds to a cost reduction of the hydrogenation reactor, which inventors evaluated to be small. Unlike the teaching of the prior art, inventors have found that, in a continuous process for producing a polyols stream comprising ethylene glycol and propylene glycol from a liquid sugar stream, there is a great advantage in conducting the first reaction at a first reaction pressure which is greater than or equal to the second reaction pressure.

Because the second reaction zone is distinct and separate from the first reaction zone, the hydrogenated mixture, which is pressurized at the first reaction pressure in the presence of the first hydrogen gas, has to be transferred from the first reaction zone to the second reaction zone.

In the case that the second reaction pressure would be greater than the first reaction pressure, corresponding to the teaching of the prior art, a first method known in the art for transferring the at least a portion of the hydrogenated mixture from the first reaction zone to the second reaction zone comprises a step of separating the hydrogenated mixture, in a liquid state, and the first hydrogen gas, which is clearly in a gas state. Typically, liquid-gas separation comprises cooling the liquid-gas mixture before performing separation and/or reducing the pressure of the liquid-gas mixture. The hydrogenated mixture and the first hydrogen gas are then separately inserted in the second reaction zone by means of two separated pumping systems, which increase the pressure of each stream to a value sufficient to permit the introduction of the streams in the second reaction zone at the second reaction pressure. The first method known in the art for transferring the hydrogenated mixture and the hydrogen gas requires devoted equipments and, according to inventors experience in operating continuous processes on industrial scale, it is not fully compatible with a continuous process.

In the case when the second reaction pressure is greater than the first reaction pressure, corresponding to the teaching of the prior art, a second method known in the art for transferring the at least a portion of the hydrogenated mixture from the first reaction zone to the second reaction zone comprises the step of increasing the pressure of the hydrogenated mixture and first hydrogen gas to a value sufficient to permit the introduction of the streams into the second reaction zone at the second reaction pressure, without liquid-gas separation. A pumping system capable of pumping a mix of pressurized liquid-gas streams would have to be used. On an industrial scale, different kinds of pumping systems are available, but they are expensive and subjected to failure.

Inventors have found that, by conducting the first reaction at a first reaction pressure which is greater than or equal to the second reaction pressure, and then releasing the hydrogen gas at the end of the conversion process of the second reaction zone at a discharge pressure which is less than the pressure of the first reaction, the transferring of at least a portion of the hydrogenated mixture and at least a portion of the first hydrogen gas from the first reaction zone to the second reaction zone may be done without the use of any pumping system, specifically of a pumping system which is capable of pumping a pressurized liquid/gas mixture.

Stated in another way, in the disclosed process the pressure does not increase along the process steps, or in two subsequent process steps, and the difference between the first reaction pressure and the discharge pressure is greater than zero.

In one preferred embodiment, the first reaction pressure is greater than the second reaction pressure. In a more preferred embodiment, the first reaction pressure is equal to the second reaction pressure. In all cases, the discharge pressure may be less than the second reaction pressure. The inventors have discovered that measured pressures of the first reaction zone and the second reaction zone are the same, even when the contents of the first reaction zone are moving the contents of the second reaction zone. This means that the difference in pressure between the two zones is immeasurable, or substantially equal. The first reaction pressure is substantially equal to the second reaction pressure, when the first reaction pressure and the second reaction pressure have the same pressure measurement and the contents of the first reaction zone are moving in a continuous manner to the second reaction zone.

Even more preferably, the first reaction pressure is a pressure at which the hydrogenolysis reaction of a sugar alcohols to at least a polyol occurs with high yield and/or high selectivity, provided that other second reaction conditions are properly defined. Stated in another way, once the second catalytic conditions comprising the second reaction pressure are defined, in the disclosed continuous process the first reaction pressure is set to be greater than or equal to the second reaction pressure, even if according to the prior art teaching the first reaction could be conducted at a pressure lower than the second reaction pressure.

In a preferred embodiment, the positive difference of pressure between the first reaction pressure and the discharge pressure generates a driving force which acts on the hydrogenated mixture and the first hydrogen gas and the transferring of the at least a portion of hydrogenated mixture and at least a portion of the first hydrogen gas from the first reaction zone and the second reaction zone occurs under the action of the driving force.

In another embodiment, the transferring of all the streams of the disclosed conversion process occurs under the action of the driving force associated to the difference of pressure between the first reaction zone and the discharge pressure.

The liquid sugar stream comprises water and at least a solubilized monomeric sugar. In a preferred embodiment the liquid sugar stream comprise xylose, glucose and arabinose, or mixture thereof.

In a more preferred embodiment, the sugars in the liquid sugar stream comprise mainly xylose and the preferred amount of xylose in the liquid sugar stream on a dry basis is greater than 50%, more preferably greater than 70%, even more preferably greater than 80%, yet even more preferably greater than 90%, being greater than 95% the most preferred value.

Even if the at least a solubilized monomeric sugar may be or may comprise a synthetic sugar, in a preferred embodiment the liquid sugar stream comprises solubilized monomeric sugars derived from a ligno-cellulosic feedstock. C5 sugars are pentose-based sugars, which may be in monomeric, oligomeric and polymeric form. Pentose is a monosaccharide with five carbon atoms. C6 sugars are hexose-based sugars, which may be in monomeric, oligomeric and polymeric form. Hexose is a monosaccharide with six carbon atoms. C5 sugars rich-streams represent a low-value stream which may be derived from the ligno-cellulosic biomass. As examples, C5 sugars rich-streams are produced as a by-product in the pulp and paper industry, or may be produced by subjecting the ligno-cellulosic feedstock to a thermal, chemical or hydrothermal process. Preferably, the liquid sugar stream is derived from the ligno-cellulosic feedstock by means of an inexpensive soaking in water, which solubilizes a portion of the carbohydrates in the feedstock without the need of chemicals or catalysts. The solid, not solubilized feedstock may then be used for feeding another conversion process or processes to different chemical end-products. The soaking of the ligno-cellulosic feedstock produces a soaked liquid stream comprising soluble sugars, mainly derived from the xylans of the ligno-cellulosic feedstock, which may be hydrolyzed to monomers and purified before feeding the process of the present disclosure. The preferred process for producing the liquid sugar stream from a ligno-cellulosic feedstock is described in details in a following section of the present specification.

Therefore, according to a further aspect of the invention, it is disclosed a process which converts a low valued liquid sugar stream to valuable chemicals reducing the conversion costs.

In an embodiment, the liquid sugar stream and the first hydrogen gas are introduced in the first reaction zone from two separated inlets. In the case that the pressure of the liquid sugar stream before entering the first reaction zone is lower that the first reaction pressure, the liquid sugar stream is first pressurized at a liquid sugar stream entry pressure, which is the pressure of the liquid sugar stream before entering the first reaction zone and is preferably greater that the first reaction pressure. Many kinds of pumping systems may be used for pumping the liquid sugar stream. A flow regulator may be used to control, or to vary, the flow of the liquid sugar stream. A valve may be used to prevent the back-flow of liquids and/or gas from the first reaction zone.

Similarly, the first hydrogen gas before entering the first reaction zone is present or pressurized at a first hydrogen gas entry pressure which is the pressure of first hydrogen gas before entering the first reaction zone and is preferably greater that the first reaction pressure. A flow regulator may be used to control, or to vary, the flow of the first hydrogen gas. A valve may be used to prevent the back-flow of liquids and/or gas from the first reaction zone.

In another embodiment, the liquid sugar stream and the first hydrogen stream are mixed together before entering the first reactor zone from a unique inlet. In the case that the liquid/gas mixture before entering the first reaction zone is at a pressure lower that the first reaction pressure, the liquid/gas mixture is first pressurized at a liquid/gas mixture entry pressure, which is the pressure of liquid/gas mixture before entering the first reaction zone and is preferably greater that the first reaction pressure. Many kinds of pumping systems may be used for pumping the liquid/gas mixture. A flow regulator may be used to control, or to vary, the flow of the liquid/gas mixture. A valve may be used to prevent the back-flow of liquids and/or gas from the first reaction zone.

In an embodiment, the liquid sugar stream and/or the first hydrogen gas, or the liquid/gas mixture are introduced without being pre-heated by means of external heating means, such as a heater or a heat exchanger.

Preferably, the liquid sugar stream and/or the first hydrogen gas, or the liquid/gas mixture are heated by means of external heating means, such as a heater or a heat exchanger to an entry temperature, which may be different for each stream. Preferably, each stream is heated to an entry temperature which is lower than the first reaction temperature. The introduction of optionally pre-heated streams is useful for not significantly disrupting the temperature in the first reaction zone which is preferably kept constant.

The liquid sugar stream and the first hydrogen gas are preferably introduced in the first reaction zone in a molar ratio corresponding to an excess of hydrogen with respect to the stoichiometric molar ratio required by the first reaction.

Preferably, the ratio of the total molar amount of monomeric sugars to the molar amount of the first hydrogen gas introduced in the first reaction zone per unit of time is a value between 1:2 to 1:10, more preferably between 1:3 and 1:8, and most preferably between 1:4: and 1:6. It is pointed out that the preferred value of the ratio of the total molar amount of monomeric sugars to the molar amount of the first hydrogen gas introduced in the first reaction zone per unit of time does not correspond to the instantaneous value of the ratio, which can be outside the preferred ranges. In the framework of the present disclosure, the ratio of the total molar amount of monomeric sugars to the molar amount of the first hydrogen gas introduced in the first reaction zone per unit of time is calculated as the arithmetic mean over a time period which is at least 50% of the first reaction time.

The first reaction zone has first reaction catalytic conditions and contains a hydrogenation catalyst, which promotes the conversion of at least a portion of the liquid sugar stream to the hydrogenated mixture comprising at least a sugar alcohol at the first reaction catalytic conditions. In the first reaction zone, the liquid sugar stream is maintained in contact with the hydrogenation catalyst. In other embodiments, depending on the specific choice of reactor and operation mode, the contact may be maintained also between the liquid sugar stream and the first hydrogen gas, or the hydrogenation catalyst and first hydrogen gas, or among the first hydrogen gas, the liquid sugar stream and hydrogenation catalyst.

The first reaction catalyst is preferably a supported metal which comprises at least a metal selected from the group of Ru, Ni and Pt, or combination thereof. The catalyst support may comprise alumina, zirconia or activated carbon, or a combination thereof. The ratio between the total amount of the sugars in the liquid sugars stream to the amount of hydrogenation catalyst is preferably between 3:2 and 3:0.5. Even if the hydrogenation catalyst may be in particle form which may be dispersed in the liquid sugar stream, in a preferred embodiment the hydrogenation catalyst is composed by particles which are aggregated to form a rigid structure, in such a way to permit liquid sugar stream and the first hydrogen gas to flow, or percolate, among catalyst particles.

The first reaction conditions comprise a first reaction pressure, a first reaction temperature and a first reaction time. The first reaction pressure may be between 40 bar to 170, preferably between 40 bar and 150 bar, more preferably between 50 bar and 100 bar, and even more preferably between 60 to 80 bar. It is noted that the preferred values of first reaction pressure are value at which the second reaction may be conducted with high yield and or selectivity.

The first reaction pressure is the actual pressure at which the first reaction is conducted. It is determined mainly by the pressure of the first hydrogen gas at the first reaction temperature, and eventually may be slightly affected by the pressure of possible gas reaction products of the first reaction and by the vapor pressure of the liquid sugar stream and liquid reaction products.

Preferably, the hydrogenating temperature is between 50° C. to 200° C., preferably between 70° C. to 150° C., more preferably between 85° C. to 130° C., and most preferably between 100 to 120° C. Even more preferably, the first reaction is conducted at a first reaction temperature promoting the conversion of all, or substantially all, the sugars in the liquid sugar stream at the first reaction pressure.

The first reaction is preferably conducted for a first reaction time sufficient for converting all, or substantially all, the monomeric sugars in the liquid sugar stream, even if a portion of the at least one monomeric sugar, which has not reacted, may be present in the hydrogenated mixture. Therefore, to be substantially converted on the basis of a monomeric sugar means that at least 90% of the monomeric sugar has been to converted on a dry weight basis. To be substantially converted on the basis of the monomeric sugars means that at least 90% of all monomeric sugars 10% or less of the monomeric sugar has been to converted on dry weight basis. The reaction time is conveniently expressed as the contact time between the liquid sugar stream and the first catalyst, in the case that the first reaction is conducted in a fixed bed configuration, or as residence time of the liquid sugar stream, in the case that the first reaction is conducted in a

Continuous Stirred-Tank Reactor configuration. The first reaction time may be a value between 20 minutes and 3 h, preferably between 25 minutes and 2 h, and most preferably between 30 minutes and 1 hour.

The hydrogenated mixture comprises water and at least a sugar alcohol.

Preferred sugar alcohols are xylitol, sorbitol and arabitol, or mixture thereof. Even more preferably, the hydrogenated mixture comprises xylitol and the preferred amount of xylitol in the hydrogenated mixture on a dry basis is greater than 45%, more preferably greater than 70%, even more preferably greater than 80%, yet even more preferably greater than 90%, being greater than 95% the most preferred value. It is noted that the hydrogenated mixture may further comprise a portion of monomeric sugars which did not reacted. A stream comprising unreacted monomeric sugars may be optionally separated the after first reaction, or in the following steps of the disclosed process, and being reused for feeding the first reaction.

In the disclosed process, at least a portion of the hydrogenated mixture and at least a portion of the first hydrogen gas are then transferred to a second reaction zone. Even if the at least a portion and the portion of the first hydrogen gas may be separated in a liquid stream, comprising the at least a portion of the hydrogenated mixture and a gas stream comprising the portion of the first hydrogen gas, in a preferred embodiment the at least a portion of the hydrogenated mixture and the portion of the first hydrogen gas are transferred to the second zone without being separated.

The at least a portion of the hydrogenated mixture and at least a portion of the first hydrogen gas may be optionally pre-heated by means of external heating means, such as a heater or a heat exchange, before entering the second reaction zone, preferable at a temperature which is lower than the second reaction temperature.

The second reaction zone has second reaction catalytic conditions and contains a hydrogenolysis catalyst, which promotes the conversion of at least a portion of the at least a sugar alcohol in the hydrogenated mixture transferred from the first reaction zone to the second zone to a hydrogenolysis mixture comprising ethylene glycol and propylene glycol at the second reaction catalytic conditions. The second reaction occurs in the presence of a second hydrogen gas which comprises the portion of the first hydrogen gas which has been transferred from the first reaction zone to the second reaction zone. In a preferred embodiment, the second hydrogen zone further comprise supplementary hydrogen which is added to the second reaction zone from an external hydrogen source. As in the case of the first hydrogen gas, the supplementary hydrogen gas before entering the second reaction zone is present or is pressurized at a supplementary hydrogen gas entry pressure which is the pressure of supplementary hydrogen gas before entering the second reaction reaction zone and is preferably greater that the second reaction pressure. A flow regulator may be used to control, or to vary, the flow of the second hydrogen gas. A valve may be used to prevent the back-flow of liquids and/or gas from the second reaction zone to the supplementary hydrogen supply line.

The hydrogenated mixture and the second hydrogen gas are preferably introduced in the second reaction zone in a molar ratio corresponding to an excess of hydrogen with respect to the stoichiometric molar ratio required by the second reaction.

Preferably, the ratio of the total molar amount of sugar alcohols to the molar amount of the second hydrogen gas introduced in the second reaction zone per unit of time is a value between 1:2 and 1:10, preferably between 1:3 and 1:8, and more preferably between 1:4: and 1:6. It is pointed out that the preferred value of the ratio of the total molar amount of sugar alcohols to the molar amount of the second hydrogen gas introduced in the second reaction zone per unit of time does not correspond to the instantaneous value of the ratio, which can be outside the preferred ranges. In the framework of the present disclosure, the ratio of the total molar amount of sugar alcohols to the molar amount of the second hydrogen gas introduced in the second reaction zone per unit of time is calculated as the arithmetic mean over a time period which is at least 50% of the second reaction time.

In the second reaction zone, the hydrogenated mixture is maintained in contact with the hydrogenolysis catalyst. In other embodiments, depending on the specific choice of reactor and operation mode, the contact may be maintained also between the hydrogenated mixture and the second hydrogen gas, or the hydrogenolysis catalyst and the second hydrogen gas, or among the second hydrogen gas, the hydrogenated mixture and the hydrogenolysis catalyst.

The hydrogenolysis catalyst comprises preferably a supported metal selected from the group of Ru, Ni and Pt, or combination thereof. The catalyst support may comprise alumina, zirconia or activated carbon, or a combination thereof. The ratio between the total amount of the sugar alcohols in the hydrogenated mixture to the amount of hydrogenolysis catalyst is preferably between 3:2 and 3:0.1. Even if the hydrogenolysis catalyst may be in particles form which may be dispersed in the hydrogenation mixture, in a preferred embodiment the hydrogenolysis catalyst is composed by particles which are aggregated to form a rigid structure, in such a way to permit the hydrogenated mixture and the second hydrogen gas to flow, or percolate, among catalyst particles.

The second reaction of the sugar alcohols occurs in the presence of OH− ions which affects the pH of the reaction environment. pH values greater than 9, corresponding to basic conditions, promote the effective hydrogenolysis of the sugar alcohols. OH− ions are preferably derived from a compound selected from the group consisting of NaOH, KOH, Ca(OH)2 and Ba(OH)2, or a combination thereof. The source of OH− ions may be introduced in the hydrogenolysis reactor or it may be added to the hydrogenated mixture before the insertion in the reactor.

The second reaction conditions comprise a second reaction pressure, a second reaction temperature and a second reaction time.

The second reaction pressure is not greater than the first reaction pressure, provided that it is sufficient to promote the conversion of at least a portion of the at least a sugar alcohol in the hydrogenated mixture at the other reaction conditions. In a preferred embodiment, the second reaction pressure is a value at which the second reaction is conducted with high yield and or selectivity at the other reaction conditions. In an even more preferred embodiment, the second reaction pressure is equal to the first reaction pressure.

The second reaction pressure is the actual pressure at which the second reaction is conducted. It is established mainly by the pressure of the second hydrogen gas at the second reaction temperature, and eventually may be slightly affected by the pressure of possible gas reaction products of the second reaction and by the vapor pressure of the hydrogenated mixture and liquid reaction products.

The second reaction temperature may be a value between 150° C. to 240° C., and most preferably between 190 to 220° C. Even more preferably, the second reaction is conducted at a second reaction temperature promoting the conversion of all, or substantially all, the sugar alcohols in the hydrogenated mixture at the second reaction pressure.

The second reaction is preferably conducted for a second reaction time sufficient for converting all, or substantially all, the sugar alcohols in the hydrogenated mixture, even if a portion of sugar alcohols, which has not reacted, may be present in the hydrogenolysis mixture. The reaction time is conveniently expressed as the contact time between the hydrogenated mixture and the second catalyst, in the case that the second reaction is conducted in a fixed bed configuration, or as residence time of the hydrogenated mixture, in the case that the second reaction is conducted in a Continuous Stirred-Tank Reactor configuration. The second reaction time may be a value between 15 minutes and 10 h, preferably between 20 minutes and 5 h, more preferably between 25 minutes and 2 hour, and most preferably between 30 minutes and 1 h.

At least a portion of the second hydrogen gas is released at a discharge pressure which is not greater, preferably less, than the first reaction pressure. Preferably, the discharge pressure is a value in a range of 0.1 bar to 30 bar, more preferably of 0.3 bar to 20 bar, even more preferably of 0.5 bar to 10 bar, even yet more preferably of 0.5 bar to 4 bar, and most preferably of 0.5 bar to 2 bar. Most preferably, the discharge pressure is substantially equal to 1 bar, which means the discharge pressure is in the range of 0.8 to 1.5 bar, with 0.9 to 1.1 bar most preferred.

In an embodiment, the releasing of the portion of the second hydrogen gas to the discharge pressure occurs without separating the portion of the second hydrogen gas and the hydrogenolysis mixture prior to releasing of the portion of the second hydrogen gas. The portion of the second hydrogen gas and at least a portion of the hydrogenolysis mixture, forming a liquid-gas mixture, may be at a pressure which is preferably not greater than, more preferably equal to, the second reaction pressure. The temperature of the liquid-gas mixture may be less than the second reaction temperature, and may be obtained by means of a heat exchange before releasing the portion of the second hydrogen gas and at least a portion of the hydrogenolysis mixture to the discharge pressure. A preferred way of releasing the portion of the second hydrogen gas, forming a liquid-gas mixture with the at least a portion of the hydrogenolysis mixture, is by means of flashing, or spraying, of the liquid-gas mixture through a nozzle valve into a separation zone, which is at a separation pressure which corresponds to the discharge pressure. The at least a portion of the liquid hydrogenolysis mixture and the portion of the second hydrogen gas are then separated in the separation zone, for instance by means of gravity and recovered separately. The removed portion of the second hydrogen gas, after an optional purification step, may be recycled in the first reaction and/or the second reaction.

In a preferred embodiment, the portion of the second hydrogen gas and the at least a portion of the hydrogenolysis mixture are separated prior to releasing the portion of the second hydrogen gas at the discharge pressure. Even if any methods may be used for the separation, in a preferred embodiment the separation occurs by means of gravity. One or more heat exchangers may be used to decrease the temperature of the liquid-gas mixture before separation or to decrease the temperature of the hydrogenolysis mixture and/or the second hydrogen gas after separation. Preferably, the liquid-gas separation occurs in a separation zone which is different from the second reaction zone and the pressure of the portion of the removed second hydrogen gas after gas-liquid separation is not greater than the second reaction pressure. The transferring of the at least a portion of the hydrogenolysis mixture and a portion of the second hydrogen from the second reaction zone to the separation zone gas may then occur without the use of a pumping system. More preferably, the transferring occurs under the action of a force associated to the difference of pressure between the first reaction zone and the discharge pressure, which may or may not correspond to a difference of pressure between the second reaction zone and the separation zone. Namely, also in the case when the pressure of the second reaction zone is the same as the pressure of the separation zone, the force associated to the difference of pressure between the first reaction zone and the discharge pressure may drive the transferring of the liquid-gas mixtures along all the reaction zone of the process. After liquid gas-separation, the portion of the second hydrogen gas which has been separated is released to the discharge pressure, preferably by means of valve, such as a nozzle valve, able to maintain the separation pressure in the separation zone which may be greater than the discharge pressure. The hydrogenolysis mixture is then recovered from the separation zone.

In a preferred embodiment, the first reaction zone is located in a first reactor and the second reaction zone is located in a second reactor. Many kind of reactors may be used for implementing the disclosed continuous process, provided that it can be operated in continuous way. Preferably, the first reactor and/or the second reactor are selected from the group consisting of fixed bed reactor, fluidized bed reactor and mechanically mixed reaction system. More preferably, the first reactor and/or the second reactor are fixed bed reactors, which may be operated as ebullating catalyst bed reactors or as a trickle bed reactors. In the most preferred embodiment, the first reactor and the second reactor are fixed bed operated as trickle bed reactors. The reactors shall be designed to be operated with different contact reaction times. Another type of reactor which may be used for implementing the first reaction zone and/or the second reaction zone is the continuous stirred tank reactor (CSTR), which is a particular type of mechanically mixed reaction system, wherein the catalyst is dispersed in the liquid reagents and products. The transferring of the at least a portion of the hydrogenated mixture and at least a portion of the first hydrogen gas from the first reactor to the second reactor may be implemented by means of many techniques, preferably without the need of a pumping system. A portion of the first catalyst may be removed from the first reactor together with the transferred mixture, and may be separated and recycled in the first reactor, optionally after a regenerative process. In a similar way, a portion of the second catalyst may be removed from the second reactor and it may be recovered for instance by means of filtration and reinserted in the second reactor, eventually after being regenerated.

In another embodiment, the first reaction zone and the second reaction zone are located in a unique reactor, which preferably is selected from the group consisting of fixed bed reactor, fluidized bed reactor and mechanically mixed reaction system. More preferably, the unique reactor is a fixed bed reactor operated as a trickle bed reactor.

Preferably, the separation zone is located in a separation device, such a s a vessel, which is separated from the first reactor and the second reactor. In a preferred embodiment, the hydrogenolysis mixture is collected in the vessel by gravity, preferably at the bottom of the vessel, and then removed from the vessel.

The hydrogenolysis reaction of the sugar alcohols in the hydrogenated mixture produces a hydrogenolysis mixture comprising water, ethylene glycol, and propylene glycol. It may further comprise glycerol and other polyols, unwanted compounds, comprising acid lactic or formic acid, and unreacted sugar alcohols.

The polyols stream comprising ethylene glycol and propylene glycol may be recovered from the hydrogenolysis mixture by any process known in the art.

In a preferred embodiment, a portion of the water of the hydrogenolysis mixture is first removed by means of a dewatering step. Dewatering may be done by thermal evaporation or by filtration. Preferably, the dry matter content of the dewatered glycols mixture is a value in the range of 40% to 95%, more preferably of 50 to 90% even more preferably of 60% to 85%, and most preferably of 70% to 80%.

The hydrogenolysis mixture, which has been eventually dewatered, is then separated into the polyols stream comprising water, ethylene glycol and propylene glycol, and at least an additional stream, comprising water, glycerol and eventually lactic acid and unreacted sugar alcohols. Other streams may be produced in the separation step. Depending on the separation conditions, the polyols stream comprising ethylene glycol and propylene glycol may further comprise other polyols, such as for instance butanediol, pentanediols, and/or other compounds which are not polyols, such as for instance unreacted or intermediates or byproducts. The additional stream may further comprise other polyols, such as threitol and erythrithol, and/or other compounds which are not polyols, such as for instance lactic acid, formic acid (which in a basic environment may be present in anionic form, such as for instance sodium lactate and sodium formate) and unreacted sugar alcohols, which are not evaporated at the separation conditions.

The preferred way for separating the hydrogenolysis mixture is by thermal evaporation, which may be conducted at a temperature between 100° C. and 140° C. and at a pressure between 30 mbar and 200 mbar, more preferably at a temperature of 120° C. and at a pressure of 50 mbar.

An ethylene glycol stream and a propylene glycol stream may be separated from the polyols mixture by means of any process known in the art, preferably by means of distillation. Optionally, other streams are produced in the separation.

The propylene glycol stream comprises 1,2-Propylene Glycol, and may further comprise ethylene glycol and/or other low boiling polyols. In the context of the present disclosure, low boiling polyols are polyols having a boiling temperature less than 300° C., at 1 bar and at a pH of the solution of 10, such as for instance butanediol and pentanediols.

The ethylene glycol stream comprises a plurality of diols, wherein ethylene glycol is the main component, as the amount of ethylene glycol, expressed as molar percent with respect to the plurality of diols, is preferably greater than 80%. In preferred embodiments, the molar amount of ethylene glycol is greater than 85%, being greater than 90% more preferable, greater than 95% even more preferable and greater than 98% the most preferable value.

In an embodiment, the ethylene glycol stream further comprises at least one diol selected from 1,2-Propylene glycol, 1,2-Butanediol and 1,2-Pentanediol.

In a preferred embodiment, the ethylene glycol stream comprises 1,2 Propylene glycol, and the percent molar amount of 1,2 Propylene glycol with respect to the plurality of diols is preferably less than 15%, more preferably less than 12%, even more preferably less than 10%, even yet more preferable less than 7%, even yet more preferable less than 5%, most preferably less than 3%, being less than 2% the even most preferred value.

In another preferred embodiment, the ethylene glycol stream comprises 1,2-Butanediol, and the percent amount of 1,2-Butanediol with respect to the plurality of diols is preferably less than 10%, more preferably less than 8%, even more preferably less than 5%, even yet more preferable less than 3%, most preferably less than 2%, being less than 1% the even most preferred value.

In a preferred embodiment, the ethylene glycol stream comprises 1,2-Pentanediol, and the percent amount of 1,2-Pentanediol with respect to the plurality of diols is preferably less than 5%, more preferably less than 4%, even more preferably less than 3%, even yet more preferable less than 2% and most preferably less than 1%.

Even if the ethylene glycol stream may comprise only one 1,2-diol, more preferably it comprises two 1,2-diols, even more preferably it comprises three 1,2-diols. Most preferably, the ethylene glycol stream comprises 1,2-Propylene glycol, 1,2-Butanediol and 1,2-Pentanediol.

The ethylene glycol stream may be used to produce a polyester resin.

A first preferred method to produce the polyester resin is the ester process, comprising an ester interchange and a polycondensation. Basically, the diols of the plurality of diols are reacted with a dicarboxylic ester (such as dimethyl terephthalate) in an ester interchange reaction, which may be catalyzed by an ester interchange catalyst. As an alcohol is formed in the reaction (methanol when dimethyl terephthalate is employed), it may be necessary to remove the alcohol to convert all or almost all of the reagents into monomers. Then monomers undergo polycondensation and the catalyst employed in this reaction is generally an antimony, germanium or titanium compound, or a mixture thereof. The ester interchange catalyst may be sequestered to prevent yellowness from occurring in the polymer by introducing a phosphorus compound, for example polyphosphoric acid, at the end of the ester interchange reaction.

A second preferred method to produce the polyester resin is the acid process, comprising a direct esterification and a polycondensation. Basically, the diols of the plurality of diols are reacted with an acid (such as terephthalic acid) by a direct esterification reaction producing monomer and water, which is removed to drive the reaction to completion. The direct esterification step does not require a catalyst. Similarly to the ester process, the monomers then undergo polycondensation to form polyester.

In both method, the polyester may be further polymerized to a higher molecular weight by a solid state polymerization, which is particularly useful for container (bottle) application.

In a preferred embodiment, at least 85% of the acid moieties of the polyester are derived from terephthalic acid or its dimethyl ester.

### Ligno-Cellulosic Feedstock

In general, a ligno-cellulosic feedstock, indicated also as ligno-cellulosic biomass can be described as follows:

Apart from starch, the three major constituents in plant biomass are cellulose, hemicellulose and lignin, which are commonly referred to by the generic term lignocellulose. Polysaccharide-containing biomasses as a generic term includes both starch and ligno-cellulosic biomasses. Therefore, some types of feedstocks can be plant biomass, polysaccharide containing biomass, and ligno-cellulosic biomass which may or may not contain starch.

Polysaccharide-containing biomasses according to the present invention include any material containing polymeric sugars e.g. in the form of starch as well as refined starch, cellulose and hemicellulose.

Relevant types of ligno-cellulosic feedstock for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all the member of the list.

In one embodiment, the ligno-cellulosic biomass feedstock used in the process is from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glumes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indian grass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

Another ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:
1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood is used to describe wood from trees that belong to the angiosperm family. Angiosperms are plants with ovules enclosed for protection in an ovary. When fertilized, these ovules develop into seeds. The hardwood trees are usually broad-leaved; in temperate and boreal latitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore, in one embodiment, a suitable ligno-cellulosic biomass may be selected from the group consisting of the grasses and woods. In one embodiment, ligno-cellulosic biomass can be selected from the group consisting of the plants belonging to the conifers, angiosperms, Poaceae and families. In one embodiment, Another preferred ligno-cellulosic biomass may be that biomass having at least 10% by weight of it dry matter as cellulose, or more preferably at least 5% by weight of its dry matter as cellulose.

Liquid Sugar Stream Derived From the Ligno-Cellulosic Feedstock

The liquid sugar stream is derived from the ligno-cellulosic feedstock by means of a treatment, or pre-treatment, of the ligno-cellulosic feedstock.

The pre-treatment of the ligno-cellulosic biomass is used to solubilize and remove carbohydrates, mainly xylans and glucans, from the ligno-cellulosic feedstock, and at the same time the concentrations of harmful inhibitory by-products such as acetic acid, furfural and hydroxymethyl furfural remain substantially low.

Pre-treatment techniques which may be used are well known in the art and include physical, chemical, and biological pre-treatment, or any combination thereof. In preferred embodiments the pre-treatment of ligno-cellulosic biomass is carried out as a batch or continuous process.

Physical pre-treatment techniques include various types of milling/comminution (reduction of particle size), irradiation

Comminution includes dry, wet and vibratory ball milling.

Although not needed or preferred, chemical pre-treatment techniques include acid, dilute acid, base, organic solvent, lime, ammonia, sulfur dioxide, carbon dioxide, pH-controlled hydrothermolysis, wet oxidation and solvent treatment.

If the chemical treatment process is an acid treatment process, it is more preferably, a continuous dilute or mild acid treatment, such as treatment with sulfuric acid, or another organic acid, such as acetic acid, citric acid, tartaric acid, succinic acid, or any mixture thereof. Other acids may also be used. Mild acid treatment means at least in the context of the invention that the treatment pH lies in the range from 1 to 5, preferably 1 to 3.

In a specific embodiment the acid concentration is in the range from 0.1 to 2.0% wt acid, preferably sulfuric acid. The acid is mixed or contacted with the ligno-cellulosic biomass and the mixture is held at a temperature in the range of around 160-220° C. for a period ranging from minutes to seconds. Specifically the pre-treatment conditions may be the following: 165-183° C., 3-12 minutes, 0.5-1.4% (w/w) acid concentration, 15-25, preferably around 20% (w/w) total solids concentration. Other contemplated methods are described in U.S. Pat. Nos. 4,880,473, 5,366,558, 5,188,673, 5,705,369 and 6,228,177.

Wet oxidation techniques involve the use of oxidizing agents, such as sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

In an embodiment both chemical and physical pre-treatment is carried out including, for example, both mild acid treatment and high temperature and pressure treatment. The chemical and physical treatment may be carried out sequentially or simultaneously.

The current strategies of thermal treatment are subjecting the ligno-cellulosic material to temperatures between 110-250° C. for 1-60 min e.g.:
Hot water extraction
Multistage dilute acid hydrolysis, which removes dissolved material before inhibitory substances are formed
Dilute acid hydrolysis at relatively low severity conditions
Alkaline wet oxidation
Steam explosion
Almost any pre-treatment with subsequent detoxification.

If a hydrothermal pre-treatment is chosen, the following conditions are preferred:

Pre-treatment temperature: 110-250° C., preferably 120-240° C., more preferably 130-230° C., more preferably 140-220° C., more preferably 150-210° C., more preferably 160-200° C., even more preferably 170-200° C. or most preferably 180-200° C.

Pre-treatment time: 1-60 min, preferably 2-55 min, more preferably 3-50 min, more preferably 4-45 min, more preferably 5-40 min, more preferably 5-35 min, more preferably 5-30 min, more preferably 5-25 min, more preferably 5-20 min and most preferably 5-15 min.

Dry matter content after pre-treatment is preferably at least 20% (w/w). Other preferable higher limits are contemplated as the amount of biomass to water in the pre-treated ligno-cellulosic feedstock be in the ratio ranges of 1:4 to 9:1; 1.3.9 to 9:1, 1:3.5 to 9:1, 1:3.25 to 9:1, 1:3 to 9:1, 1:2.9 to 9:1, 1:2 to 9:1, 1.15 to 9:1, 1:1 to 9:1, and 1:0.9 to 9:1.

A preferred embodiment of the process used for deriving the liquid sugar stream from the ligno-cellulosic biomass and comprises a pre-treatment and other process steps.

A preferred pre-treatment of a ligno-cellulosic biomass include a soaking of the ligno-cellulosic biomass feedstock and optionally a steam explosion of at least a part of the soaked ligno-cellulosic biomass feedstock.

The soaking occurs in a substance such as water in either vapor form, steam, or liquid form or liquid and steam together, to produce a product. The product is a soaked biomass containing a soaking liquid, with the soaking liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

Either soaking step could also include the addition of other compounds, e.g. H2SO4, NH3, in order to achieve higher performance later on in the process.

The product comprising the soaking liquid, or soaked liquid, is then passed to a separation step where at least a portion of the soaking liquid is separated from the soaked biomass. The liquid will not completely separate so that at least a portion of the soaking liquid is separated, with preferably as much soaking liquid as possible in an economic time frame. The liquid from this separation step is known as the soaked liquid stream comprising the soaking liquid. The soaked liquid will be the liquid used in the soaking, generally water and the soluble species of the feedstock. These water soluble species comprise glucan, xylan, galactan, arabinan, and their monomers and oligomers. The solid biomass is called the first solid stream as it contains most, if not all, of the solids.

The separation of the soaked liquid can again be done by known techniques. A preferred piece of equipment is a press, as a press will generate a liquid under high pressure.

The first solid stream may then optionally be steam exploded to create a steam exploded stream, comprising solids. Steam explosion is a well-known technique in the biomass field and any of the systems available today and in the future are believed suitable for this step. The severity of the steam explosion is known in the literature as Ro, and is a function of time and temperature and is expressed as

Ro=texp[(T−100)/14.75]
with temperature, T expressed in Celsius and time, t, expressed in minutes.

The formula is also expressed as Log(Ro), namely

Log(Ro)=Ln(t)+[(T−100)/14.75].

Log(Ro) is preferably in the ranges of 2.8 to 5.3, 3 to 5.3, 3 to 5.0 and 3 to 4.3.

The steam exploded stream may be optionally washed at least with water and there may be other additives used as well. It is conceivable that another liquid may be used for the same purpose, so water is not believed to be absolutely essential. At this point, water is the preferred liquid. The liquid effluent from the optional wash may be added to the soaked liquid stream. This wash step is not considered essential and is optional.

The washed exploded stream is then processed to remove at least a portion of the liquid in the washed exploded material. This separation step is also optional. The term at least a portion is removed, is to remind one that while removal of as much liquid as possible is desirable (preferably by pressing), it is unlikely that 100% removal is possible. In any event, 100% removal of the water is not desirable since water is needed for the subsequent hydrolysis reaction. The preferred process for this step is again a press, but other known techniques may be suitable. The liquid products separated from this process may be added to the soaked liquid stream.

In an embodiment, the ligno-cellulosic biomass is exposed to a presoaking step before a soaking step in a temperature range of between 10° C. and 150° C., 25° C. to 150° C. even more preferable, with 25° C. to 145° C. even more preferable, and 25° C. to 100° C. and 25° C. to 90° C. also being preferred ranges.

The pre-soaking time could be lengthy, such as up to but preferably less than 48 hours, or less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

The pre-soaking step is done in the presence of a liquid which is the pre-soaked liquid. After soaking, this liquid preferably has removed less than 5% by weight of the total sugars in the raw material, more preferably, less than 2.5% by weight of the total sugars in the raw material being more preferable, with less than 1% by weight of the total sugars in the raw material, being the most preferred.

This pre-soaking step is useful as a modification to the soaking step of a biomass pre-treatment step. In soaking (not pre-soaking) of the biomass pre-treatment steps, the soaked liquid stream which has been separated from the soaked solids will preferably have reduced filter plugging components so that the soaked liquid can be easily purified, preferably by means of at least one technique selected from the group of chromatography, nanofiltration and ultrafiltration. The soaked liquid stream may be subjected to more than one purification step, which may be done before hydrolysis or decationization.

The soaked liquid stream will comprise water, sugars which includes monomeric sugars and oligomeric sugars, salts which are dissociated into anions and cations in the soaked liquid stream, optionally phenols, furfural, oils and acetic acid. The soaked liquid stream will in particular contain xylooligomers.

Ideally, the concentration of the total sugars in the soaked liquid stream should be in the range of 0.1 to 300 g/l, with 50 to 290 g/l being most preferred, and 75 to 280 g/l even more preferred, with 100 to 250 g/l most preferred. This concentration can be done by the removal of water. A 50% removal of water increases the concentration of the non-water species by two. While various concentration increases are acceptable, in one embodiment, at least a two fold increase in the concentration of the xyloligomers in the soaked liquid stream is reached. In one embodiment, at least a fourfold increase in the concentration of the xyloligomers in the soaked liquid stream is reached. In one embodiment, at least a six fold increase in the concentration of the xyloligomers in the soaked liquid stream is reached. Many concentration steps may be applied to the soaked liquid stream before or after each process step.

In a preferred embodiment, the soaked liquid stream is subjected to hydrolysis for converting at least a portion of the oligomers in the soaked liquid stream to monomers. Hydrolysis of oligomers may be obtained by contacting the soaked liquid stream with a hydrolysis catalyst at hydrolysis conditions. The hydrolysis catalyst may be an inorganic acid, such as sulfuric acid, or an enzyme or enzyme cocktail. The hydrolysis conditions will vary according to the selected hydrolysis catalyst, and are well known in the art.

A preferred way to conduct the hydrolysis of the soaked liquid stream comprises at least two steps, according to the teaching of WO2013026849. The first step is to create an acidic stream from the soaked liquid stream. This is accomplished by increasing the amount of H+ ions to the soaked liquid stream to create the acidic stream. After the desired pH is obtained, the next step is hydrolyzing the oligosaccharides in the acidic stream by raising the temperature of the acidic stream to a hydrolysis temperature for the hydrolysis reaction to occur creating a hydrolyzed stream.

While the creation of the acidic stream can be done in any manner which increases the concentration of H+ ions, a preferred embodiment is to take advantage of the salt content of the soaked liquid stream. In order to obtain the required acidity for the hydrolysis step, the content of salts in the soaked liquid stream can be reduced via cation exchange while at the same time replacing the cations with H+ ions. While the salts may naturally occur in the soaked liquid stream, they can also be added as part of the pre-treatment processes or prior to or during the creation of the acidic stream.

In one embodiment, the hydrolyzed stream is a cleaner liquid, containing almost exclusively monomeric sugars, low content of salts and low amount of degradation products that could hinder subsequent chemical or biological transformations of the sugars.

In a preferred embodiment, the liquid sugar stream comprises at least a portion of the hydrolyzed stream.

In another preferred embodiment, the liquid sugar stream is comprised of at least a portion of the hydrolyzed stream.

1-32. (canceled)

33. A continuous process for producing a polyols stream comprising ethylene glycol and propylene glycol, wherein the process comprises the steps of:
a. introducing a first hydrogen gas and a liquid sugar stream, said liquid sugar stream comprising water and at least a monomeric sugar, into a first reaction zone, wherein the first reaction zone contains a hydrogenation catalyst and has a first reaction pressure and a first reaction temperature;
b. maintaining the liquid sugar stream in the first reaction zone in contact with at least the hydrogenation catalyst for a first reaction time sufficient to produce an hydrogenated mixture comprising at least a sugar alcohol derived from the monomeric sugar;
c. transferring at least a portion of the hydrogenated mixture and at least a portion of the first hydrogen gas from the first reaction zone to a second reaction zone, wherein the second reaction zone has a second reaction pressure which is not greater than the first reaction pressure and a second reaction temperature and further contains a hydrogenolysis catalyst capable of converting the sugar alcohol to at least a polyol at the second reaction pressure and the second reaction temperature;
d. maintaining the at least a portion of the hydrogenated mixture in the second reaction zone in contact with the hydrogenolysis catalyst in the presence of OH⁻ ions and a second hydrogen gas comprising the at least a portion of the first hydrogen gas for a second reaction time sufficient to produce an hydrogenolysis mixture comprising the polyols;
e. releasing at least a portion of the second hydrogen gas at a discharge pressure, wherein the discharge pressure is not greater than the second pressure; and
f. recovering at least a portion of the polyols from at least a portion of the hydrogenolysis mixture to form the polyols stream,
wherein the first reaction pressure is greater than the discharge pressure.

34. The process of claim 33, wherein the step c) of transferring at least a portion of the hydrogenated mixture and at least a portion of the first hydrogen gas from the first reaction zone to the second reaction zone occurs without the use of a pumping system.

35. The process of claim 33, wherein the transferring of at least a portion of the hydrogenated mixture and at least a portion of the first hydrogen gas from the first reaction zone to the second reaction zone occurs under the action of a driving force which is the force associated to the difference of the first reaction pressure and the discharge pressure.

36. The process of claim 33, wherein the first reaction pressure is a value in a range selected from the group consisting of 40 bar to 170 bar, 40 bar to 150 bar, 50 bar to 100 bar, and 60 bar to 80 bar.

37. The process of claim 33, wherein the discharge pressure is a value in a range of 0.1 bar to 30 bar, 0.3 bar to 20 bar, 0.5 bar to 10 bar, 0.5 bar to 4 bar, and 0.5 bar to 2 bar.

38. The process of claim 33, further comprising the step of separating the at least a portion of the hydrogenolysis mixture and the at least a portion of the second hydrogen gas before step e) of releasing the at least a portion of the second hydrogen gas.

39. The process of claim 38, wherein the separation of the at least a portion of the hydrogenolysis mixture and the at least a portion of the second hydrogen gas occurs in a separation zone, wherein the separation zone has a separation pressure which is less than or equal to the second reaction pressure and greater than the discharge pressure.

40. The process of claim 39, wherein the at least a portion of the hydrogenolysis mixture and the at least a portion of the second hydrogen gas are transferred from the second reaction zone to the separation zone without the use of a pumping system.

41. The process of claim 39, wherein the transferring of the at least a portion of the hydrogenolysis mixture and at least a portion of the second hydrogen gas from the second reaction zone to a separation zone occurs under the action of a driving force which is the force associated to the difference of the first reaction pressure and the discharge pressure.

42. The process of claim 33, wherein the liquid sugar stream and the first hydrogen gas are mixed together before being inserted in the first reaction zone.

43. The process of claim 33, wherein the ration of the total molar amount of monomeric sugars to the molar amount of the first hydrogen gas introduced in the first reaction zone per unit of time is a value in a range selected from the group consisting of 1:2 to 1:10, 1:3 to 1:8, and 1:4 to 1:6.

44. The process of claim 33, wherein the second hydrogen gas further comprises hydrogen added to the second reaction zone from an external hydrogen source.

45. The process of claim 33, wherein the ration of the total molar amount of sugar alcohols to the molar amount of the second hydrogen gas introduced in the second reaction zone is a value in a range selected from the group consisting of 1:2 to 1:10, 1:3 to 1:8, and 1:4 to 1:6.

46. The process of claim 33, wherein the first reaction time is a value in a range selected from the group consisting of 20 minutes to 3 hours, 25 minutes to 2 hours, and 30 minutes to 1 hour.

47. The process of claim 33, wherein the second reaction time is a value in a range selected from the group consisting of 15 minutes to 10 hours, 20 minutes to 5 hours, 25 minutes to 2 hours, and 30 minutes to 1 hour.

48. The process of claim 33, wherein the first reaction zone is located in a first reactor and the second reaction zone is located in a second reactor.

49. The process of claim 48, wherein the first reactor and/or the second reactor are selected from the group consisting of fixed bed reactor, fluidized bed reactor and mechanically mixed reaction systems.

50. The process of claim 49, wherein the first reactor and/or the second reactor are a fixed bed reactor operated as a trickle bed reactor.

51. The process of claim 33, wherein the first reaction zone and the second reaction zone are located in a unique reactor.

52. The process of claim 51, wherein the unique reactor is selected from the group consisting of fixed bed reactor, fluidized bed reactor and mechanically mixed reaction system.

53. The process of claim 52, wherein the unique reactor is a fixed bed reactor operated as a trickle bed reactor.

54. The process of claim 48, wherein the separation zone is located in a separation vessel.

55. The process of claim 33, wherein the at least a monomeric sugar comprises at least a compound selected from the group consisting of xylose, glucose and arabinose, or mixture thereof.

56. The process of claim 55, wherein the at least a monomeric sugar comprises xylose and the percent amount of xylose by weight on a dry matter basis in the liquid sugar stream is greater than a value selected from the group consisting of 50%, 70%, 80%, 90% and 95%.

57. The process of claim 55, wherein at least a portion of the monomeric sugars has been obtained from a liquid stream comprising solubilized C5 and C6 sugars which has been removed from a ligno-cellulosic biomass feedstock stream.

58. The process of claim 57, wherein the amount of solubilized C5 sugars in the liquid stream is greater than the amount of solubilized C6 sugars.

59. The process of claim 33, wherein the at least a sugar alcohol comprises a compound selected from the group consisting of xylitol, sorbitol and arabitol, or mixture thereof.

60. The process of claim 33, wherein at least a portion of the ethylene glycol and at least a portion of the propylene glycol in the polyols stream are separated from polyols stream to produce an ethylene glycol stream comprising ethylene glycol and a propylene glycol stream comprising propylene glycol.

61. The process of claim 60, wherein the ethylene glycol stream further comprises at least one diol selected from the group consisting of 1,2-Propylene glycol, 1,2-Butanediol and 1,2-Pentanediol.

62. The process of claim 60, further comprising processing the ethylene glycol stream for producing a polyester resin.

63. The process of claim 62, wherein the polyester comprises acid moieties and at least 85 mole % of the acid moieties are derived from terephthalic acid or its dimethyl ester.

64. The process of claim 62, further comprising process the polyester resin for producing a polyester bottle.
